(19) United States
(12) Patent Application Publication
(10) Pub. No.: US 2026/0318866 A1
(43) Pub. Date: Oct. 15, 2026

(54) GAS SENSOR AND SENSOR ELEMENT

(71) Applicant: NGK INSULATORS, LTD., Nagoya-shi (JP)

(72) Inventors: Shingo TANAKA, Kasugai-shi (JP); Yusuke WATANABE, Nagoya-shi (JP)

(21) Appl. No.: 19/466,770

(22) Filed: Feb. 2, 2026

(30) Foreign Application Priority Data
Feb. 4, 2025 (JP) 2025-016602

(51) Int. Cl. G01N 27/41; G01N 33/00

(52) U.S. Cl. CPC G01N 27/41; G01N 33/0027

(57) ABSTRACT

A gas sensor includes a sensor element and a control device, and configured to measure a water concentration and/or a carbon dioxide concentration in a measurement gas. The control device performs a water concentration measurement processing for measuring the water concentration in the measurement gas based on a second pump current flowing through a second pump cell by a second pump cell control processing, and the sensor element satisfies that a ratio Ih/Ir of a limiting current Ih to a limiting current Ir is 0.44 or less; and/or the control device performs a carbon dioxide concentration measurement processing for measuring the carbon dioxide concentration in the measurement gas based on a third pump current flowing through a third pump cell by a third pump cell control processing, and the sensor element satisfies that a ratio Ic/Ir of a limiting current Ic to the limiting current Ir is 0.24 or less.

### CROSS-REFERENCE TO RELATED APPLICATION

This application claims priority based on Japanese Patent Application No. 2025-016602, filed on Feb. 4, 2025, and the entire contents of which is incorporated herein by reference.

### BACKGROUND OF THE INVENTION

### 1. Field of the Invention

The present invention relates to a gas sensor and a sensor element.

### 2. Description of the Related Art

Hitherto, gas sensor that measure a water concentration and a carbon dioxide concentration in a measurement gas such as exhaust gas from an automobile have been known. For example, PTL 1 describes a gas sensor comprising a sensor element having an oxygen-ion-conductive solid electrolyte layer and provided with a gas flow path therein, which measures the concentrations of water vapor component and carbon dioxide component in the measurement gas. The gas flow path is formed so that a gas inlet, a first diffusion rate-limiting section, a first internal cavity, a second diffusion rate-limiting section, and a second internal cavity are communicated in this order. A main pump cell is configured including a main inner pump electrode disposed in the first internal cavity and an outer pump electrode disposed on an outer surface of the sensor element. A first measurement pump cell is configured including a first measurement inner pump electrode disposed in the second internal cavity and the outer pump electrode. A second measurement pump cell is configured including a second measurement inner pump electrode disposed on the opposite side of the second diffusion rate-limiting section with respect to the first measurement inner pump electrode, and the outer pump electrode. In this gas sensor, an oxygen partial pressure in the first internal cavity is adjusted by the main pump cell so that substantially all of the water vapor component and the carbon dioxide component in the measurement gas are decomposed in the first internal cavity. Then, oxygen is supplied to the second internal cavity by the first measurement pump cell so that hydrogen generated by a decomposition of the water vapor component is selectively burned (oxidized) in the second internal cavity, and the concentration of the water vapor component present in the measurement gas is measured based on a magnitude of a current flowing then. In addition, oxygen is supplied to a vicinity of a surface of the second measurement inner pump electrode by the second measurement pump cell so that carbon monoxide generated by a decomposition of the carbon dioxide component is selectively burned (oxidized) in the vicinity of the surface of the second measurement inner pump electrode, and the concentration of the carbon dioxide component present in the measurement gas is measured based on a magnitude of a current flowing then.

### CITATION LIST

### Patent Literature

PTL 1: JP 5918177 B

### SUMMARY OF THE INVENTION

In such a gas sensor, when the water concentration in the measurement gas changes, there has been a demand to shorten the time until a current flowing through the first measurement pump cell reaches a value corresponding to a changed water concentration, i.e., to improve the responsiveness of water concentration measurement by the gas sensor. Similarly, there has been a demand to improve the responsiveness of carbon dioxide concentration measurement by the gas sensor.

The present invention was made to solve such a problem, and its main object is to improve the responsiveness of measuring a water concentration and/or a carbon dioxide concentration.

The present invention employs the following configuration to achieve the above-described main object.
[1]A first gas sensor according to the present invention is a gas sensor including a sensor element and a control device, and configured to measure a water concentration and/or a carbon dioxide concentration in a measurement gas, wherein the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; a second pump cell including a second inner electrode disposed in a second chamber located downstream of the first chamber of the measurement gas flow path, and a second outer electrode disposed on an outer surface of the element body; and a third pump cell including a third inner electrode disposed in a third chamber located downstream of the second chamber of the measurement gas flow path, and a third outer electrode disposed on an outer surface of the element body; the control device performs: a first pump cell control processing in which oxygen is pumped out from around the first inner electrode to around the first outer electrode by controlling the first pump cell, thereby reducing water and carbon dioxide in the measurement gas in the first chamber; a second pump cell control processing in which oxygen is pumped from around the second outer electrode to around the second inner electrode by controlling the second pump cell, thereby oxidizing hydrogen generated by the reduction of water in the first chamber in the second chamber; and a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], a limiting current at the time the second pump cell pumps out oxygen from around the second inner electrode to around the second outer electrode is defined as a limiting current Ih [mA], and a limiting current at the time the third pump cell pumps out oxygen from around the third inner electrode to around the third outer electrode is defined as a limiting current Ic [mA], the control device performs a water concentration measurement processing for measuring the water concentration in the measurement gas based on a second pump current flowing through the second pump cell by the second pump cell control processing, and the sensor element satisfies that a ratio Ih/Ir of the limiting current Ih to the limiting current Ir is 0.44 or less; and/or the control device performs a carbon dioxide concentration measurement processing for measuring the carbon dioxide concentration in the measurement gas based on a third pump current flowing through the third pump cell by the third pump cell control processing, and the sensor element satisfies that a ratio Ic/Ir of the limiting current Ic to the limiting current Ir is 0.24 or less.

In this first gas sensor, the water concentration measurement processing is performed to measure the water concentration in the measurement gas based on the second pump current flowing through the second pump cell by the second pump cell control processing, and/or the carbon dioxide concentration measurement processing is performed to measure the carbon dioxide concentration in the measurement gas based on the third pump current flowing through the third pump cell by the third pump cell control processing. Thus, this gas sensor measures the water concentration and/or the carbon dioxide concentration in the measurement gas. Furthermore, when this gas sensor is a sensor that performs the water concentration measurement processing, the sensor element is configured such that the ratio Ih/Ir of the limiting current Ih to the limiting current Ir is 0.44 or less. In this manner, the responsiveness of water concentration measurement can be improved. When this gas sensor is a sensor that performs the carbon dioxide concentration measurement processing, the sensor element is configured such that the ratio Ic/Ir of the limiting current Ic to the limiting current Ir is 0.24 or less. In this manner, the responsiveness of carbon dioxide concentration measurement can be improved. The inventors have confirmed these effects through experiments and analyses. The reason why the responsiveness of water concentration measurement can be improved by setting Ih/Ir to 0.44 or less is considered as follows. First, the limiting current Ir has a negative correlative relationship with a diffusion resistance from outside the sensor element to the first chamber of the measurement gas flow path. The limiting current Ih has a negative correlative relationship with a diffusion resistance from outside the sensor element to the second chamber of the measurement gas flow path. Therefore, the larger the ratio Ih/Ir, the smaller the proportion of water that flows into the first chamber from outside, and the larger the proportion of water that back-diffuses from the second chamber to the first chamber, among the water presents in the measurement gas in the first chamber. As a result, a discrepancy is likely to occur between an amount of hydrogen generated by the reduction of water in the first chamber by the first pump cell control processing and an amount of water in the measurement gas outside the sensor element. Consequently, when the water concentration in the measurement gas outside the sensor element changes, the time until the amount of hydrogen generated by the reduction of water in the first chamber corresponds to the changed water concentration becomes longer, and hence the time until the second pump current flowing through the second pump cell by the second pump cell control processing corresponds to the changed water concentration becomes longer. For these reasons, it is considered that the larger the ratio Ih/Ir, the more the responsiveness of water concentration measurement tends to decrease. By setting Ih/Ir to 0.44 or less, the proportion of water that back-diffuses from the second chamber to the first chamber among the water in the measurement gas in the first chamber can be sufficiently reduced, which is considered to improve the responsiveness of water concentration measurement. The reason why the responsiveness of carbon dioxide concentration measurement can be improved by setting Ic/Ir to 0.24 or less is considered in the same manner. The larger the ratio Ic/Ir, the smaller the proportion of carbon dioxide that flows into the first chamber from outside, and the larger the proportion of carbon dioxide that back-diffuses from the third chamber to the first chamber, among the carbon dioxide presents in the measurement gas in the first chamber. As a result, a discrepancy is likely to occur between an amount of carbon monoxide generated by the reduction of carbon dioxide in the first chamber by the first pump cell control processing and an amount of carbon dioxide in the measurement gas outside the sensor element. Consequently, when the carbon dioxide concentration in the measurement gas outside the sensor element changes, the time until the amount of carbon monoxide generated by the reduction of carbon dioxide in the first chamber corresponds to the changed carbon dioxide concentration becomes longer, and hence the time until the third pump current flowing through the third pump cell by the third pump cell control processing corresponds to the changed carbon dioxide concentration becomes longer. For these reasons, the larger the ratio Ic/Ir, the more the responsiveness of carbon dioxide concentration measurement tends to decrease. By setting Ic/Ir to 0.24 or less, the proportion of carbon dioxide that back-diffuses from the third chamber to the first chamber among the carbon dioxide in the measurement gas in the first chamber can be sufficiently reduced, which is considered to improve the responsiveness of carbon dioxide concentration measurement. Note that the upper limits for improving responsiveness differ between Ih/Ir and Ic/Ir presumably because the diffusion rates of water (and hydrogen) and carbon dioxide (and carbon monoxide) are different.
[2] In the above gas sensor (the gas sensor according to [1]), the control device may perform the water concentration measurement processing, and the limiting current Ih may be 0.3 mA or less; and/or the control device may perform the carbon dioxide concentration measurement processing, and the limiting current Ic may be 0.3 mA or less. If the limiting current Ih is too large, i.e., if the diffusion resistance from outside the sensor element to the second chamber is too small, the flow rate of hydrogen flowing into the second chamber may become excessively large, resulting in insufficient oxidation of hydrogen by the second pump cell control processing. Consequently, the second pump current may no longer accurately correspond to the water concentration, leading to a decrease in the accuracy of water concentration measurement. If Ih is 0.3 mA or less, such a decrease in measurement accuracy can be suppressed. Similarly, if Ic is 0.3 mA or less, insufficient oxidation of carbon monoxide in the third chamber by the third pump cell control processing can be suppressed, thereby suppressing a decrease in the accuracy of carbon dioxide concentration measurement.
[3] In the above gas sensor (the gas sensor according to [1] or [2]), the control device may perform the water concentration measurement processing and the carbon dioxide concentration measurement processing, and the sensor element may satisfy that the ratio Ih/Ir is 0.44 or less and the ratio Ic/Ir is 0.24 or less. In this way, the responsiveness of both water concentration measurement and carbon dioxide concentration measurement can be improved.
[4]A second gas sensor according to the present invention is a gas sensor including a sensor element and a control device, and configured to measure a water concentration in a measurement gas, wherein the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; and a second pump cell including a second inner electrode disposed in a second chamber located downstream of the first chamber of the measurement gas flow path, and a second outer electrode disposed on an outer surface of the element body; the control device performs: a first pump cell control processing in which oxygen is pumped out from around the first inner electrode to around the first outer electrode by controlling the first pump cell, thereby reducing water in the measurement gas in the first chamber; a second pump cell control processing in which oxygen is pumped from around the second outer electrode to around the second inner electrode by controlling the second pump cell, thereby oxidizing hydrogen generated by the reduction of water in the first chamber in the second chamber; and a water concentration measurement processing for measuring the water concentration in the measurement gas based on a second pump current flowing through the second pump cell by the second pump cell control processing; and in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the second pump cell pumps out oxygen from around the second inner electrode to around the second outer electrode is defined as a limiting current Ih [mA], the sensor element satisfies that a ratio Ih/Ir of the limiting current Ih to the limiting current Ir is 0.44 or less.

In this second gas sensor, similarly to the above first gas sensor, by setting Ih/Ir to 0.44 or less, the responsiveness of water concentration measurement can be improved.
[5]A third gas sensor according to the present invention is a gas sensor including a sensor element and a control device, and configured to measure a carbon dioxide concentration in a measurement gas, wherein the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; and a third pump cell including a third inner electrode disposed in a third chamber located downstream of the first chamber of the measurement gas flow path, and a third outer electrode disposed on an outer surface of the element body; the control device performs: a first pump cell control processing in which oxygen is pumped out from around the first inner electrode to around the first outer electrode by controlling the first pump cell, thereby reducing carbon dioxide in the measurement gas in the first chamber; a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and a carbon dioxide concentration measurement processing for measuring the carbon dioxide concentration in the measurement gas based on a third pump current flowing through the third pump cell by the third pump cell control processing; and in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the third pump cell pumps out oxygen from around the third inner electrode to around the third outer electrode is defined as a limiting current Ic [mA], a ratio Ic/Ir of the limiting current Ic to the limiting current Ir is 0.24 or less.

In this third gas sensor, similarly to the above first gas sensor, by setting Ic/Ir to 0.24 or less, the responsiveness of carbon dioxide concentration measurement can be improved.
[6]A first sensor element according to the present invention is a sensor element for measuring a water concentration and/or a carbon dioxide concentration in a measurement gas, the sensor element including: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; a second pump cell including a second inner electrode disposed in a second chamber located downstream of the first chamber of the measurement gas flow path, and a second outer electrode disposed on an outer surface of the element body; and a third pump cell including a third inner electrode disposed in a third chamber located downstream of the second chamber of the measurement gas flow path, and a third outer electrode disposed on an outer surface of the element body; wherein, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], a limiting current at the time the second pump cell pumps out oxygen from around the second inner electrode to around the second outer electrode is defined as a limiting current Ih [mA], and a limiting current at the time the third pump cell pumps out oxygen from around the third inner electrode to around the third outer electrode is defined as a limiting current Ic [mA], a ratio Ih/Ir of the limiting current Ih to the limiting current Ir is 0.44 or less and/or a ratio Ic/Ir of the limiting current Ic to the limiting current Ir is 0.24 or less.

This sensor element has Ih/Ir of 0.44 or less and/or Ic/Ir of 0.24 or less. Therefore, when Ih/Ir of this sensor element is 0.44 or less, this sensor element is suitable as the sensor element of a gas sensor (e.g., the above first gas sensor) that performs water concentration measurement. When Ic/Ir is 0.24 or less, this sensor element is suitable as the sensor element of a gas sensor (e.g., the above first gas sensor) that performs carbon dioxide concentration measurement.
[7]A second sensor element according to the present invention is a sensor element for measuring a water concentration in a measurement gas, the sensor element including: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; and a second pump cell including a second inner electrode disposed in a second chamber located downstream of the first chamber of the measurement gas flow path, and a second outer electrode disposed on an outer surface of the element body; wherein, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the second pump cell pumps out oxygen from around the second inner electrode to around the second outer electrode is defined as a limiting current Ih [mA], a ratio Ih/Ir of the limiting current Ih to the limiting current Ir is 0.44 or less.

This sensor element has Ih/Ir of 0.44 or less. Therefore, this sensor element is suitable as the sensor element of a gas sensor (e.g., the above second gas sensor) that performs water concentration measurement.
[8]A third sensor element according to the present invention is a sensor element for measuring a carbon dioxide concentration in a measurement gas, the sensor element including: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; and a third pump cell including a third inner electrode disposed in a third chamber located downstream of the first chamber of the measurement gas flow path, and a third outer electrode disposed on an outer surface of the element body; wherein, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the third pump cell pumps out oxygen from around the third inner electrode to around the third outer electrode is defined as a limiting current Ic [mA], a ratio Ic/Ir of the limiting current Ic to the limiting current Ir is 0.24 or less.

This sensor element has Ic/Ir of 0.24 or less. Therefore, this sensor element is suitable as the sensor element of a gas sensor (e.g., the above third gas sensor) that performs carbon dioxide concentration measurement.

### BRIEF DESCRIPTION OF THE DRAWINGS

FIG. 1 is a schematic cross-sectional view schematically showing an example of a configuration of a gas sensor 100.

FIG. 2 is a block diagram showing the electrical connections between a control device 95 and respective cells and other components.

FIG. 3 is a graph showing a relationship between the ratio Ih/Ir and a response time Th0 for each of Experimental Examples 1 to 10.

FIG. 4 is a graph showing a relationship between the ratio Ih/Ir and a response time Th1 for each of Experimental Examples 1 to 10.

FIG. 5 is a graph showing a relationship between the ratio Ic/Ir and a response time Tc0 for each of Experimental Examples 11 to 20.

FIG. 6 is a graph showing a relationship between the ratio Ic/Ir and a response time Tc2 for each of Experimental Examples 11 to 20.

FIG. 7 is a schematic cross-sectional view of a sensor element 201 according to a modification.

FIG. 8 is a schematic cross-sectional view schematically showing an example of a configuration of a gas sensor 300 according to a modification.

FIG. 9 is a schematic cross-sectional view schematically showing an example of a configuration of a gas sensor 400 according to a modification.

### DETAILED DESCRIPTION OF THE INVENTION

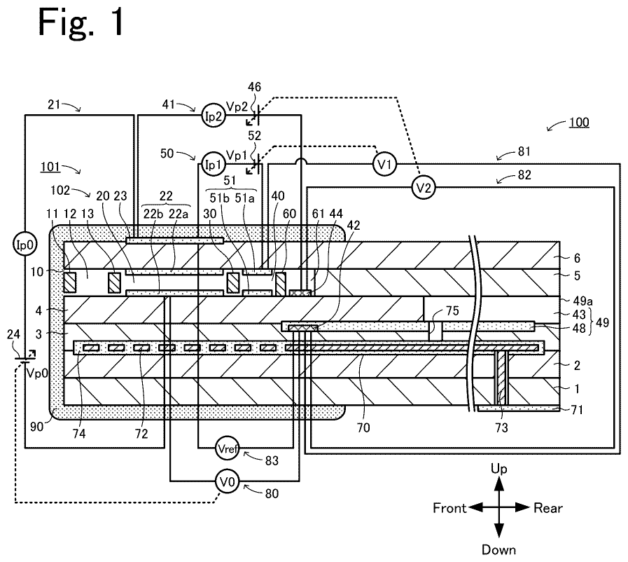

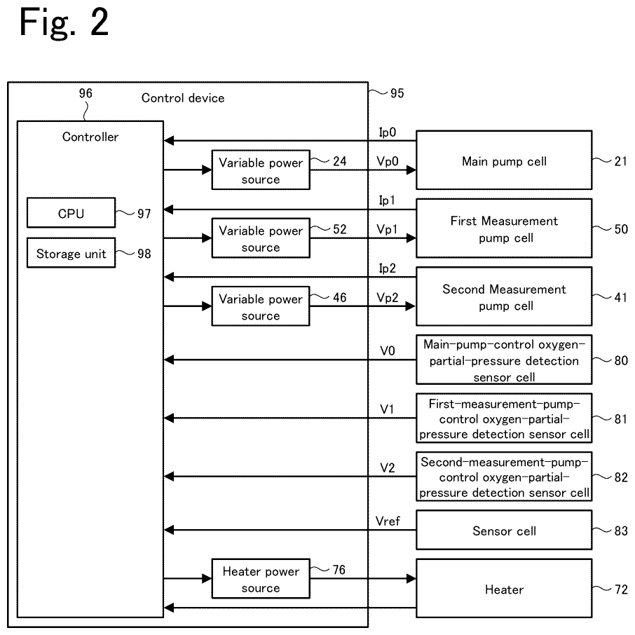

Next, embodiments of the present invention will be described with reference to the drawings. FIG. 1 is a schematic cross-sectional view schematically showing an example of a configuration of a gas sensor 100 according to an embodiment of the present invention. FIG. 2 is a block diagram showing the electrical connections between a control device 95, respective cells and a heater 72. The gas sensor 100 is installed in a pipe, such as an exhaust pipe of an internal combustion engine. The gas sensor 100 measures a concentration of a specific gas in a measurement gas, using exhaust gas from an internal combustion engine as the measurement gas. In the present embodiment, the gas sensor 100 is configured to detect the water concentration and the carbon dioxide concentration as the concentrations of the specific gases. The gas sensor 100 includes: a sensor element 101 with an elongated rectangular parallelepiped element body 102; cells 21, 41, 50, and 80 to 83 within the sensor element 101; a heater section 70 provided inside the sensor element 101; a porous protective layer 90 provided in the sensor element 101; and a control device 95, which includes variable power sources 24, 46, and 52, and a heater power source 76, and controls the overall operation of the gas sensor 100. Note that the longitudinal direction (left-right direction in FIG. 1) of the sensor element 101 is defined as the front-rear direction, the thickness direction (up-down direction in FIG. 1) of the sensor element 101 as the up-down direction, and the width direction (perpendicular to both front-rear direction and up-down direction) of the sensor element 101 as the left-right direction.

The element body 102 is a laminated body in which six layers are stacked in the following order from the bottom in the drawing: a first substrate layer 1, a second substrate layer 2, a third substrate layer 3, a first solid electrolyte layer 4, a spacer layer 5, and a second solid electrolyte layer 6. Each of these layers is composed of an oxygen-ion-conductive solid electrolyte layer, such as zirconia (ZrO₂) or the like. The solid electrolytes forming these six layers are dense and hermetically sealed. The element body 102 is manufactured, for example, by performing predetermined processing and printing of circuit patterns on ceramic green sheets corresponding to the respective layers, laminating the sheets, and then firing the laminated sheets to integrate them into a unified structure.

On the front end side of the sensor element 101 (element body 102), between the lower surface of the second solid electrolyte layer 6 and the upper surface of the first solid electrolyte layer 4, the following components are formed adjacently and connected in sequence: a gas inlet 10; a first diffusion rate-limiting section 11; a buffer space 12; a second diffusion rate-limiting section 13; a first internal cavity 20; a third diffusion rate-limiting section 30; a second internal cavity 40; a fourth diffusion rate-limiting section 60; and a third internal cavity 61.

The gas inlet 10, buffer space 12, first internal cavity 20, second internal cavity 40, and third internal cavity 61 are internal spaces within the sensor element 101, formed by hollowing out portions of the spacer layer 5. These spaces are bounded at the top by the lower surface of the second solid electrolyte layer 6, at the bottom by the upper surface of the first solid electrolyte layer 4, and on the sides by the side surfaces of the spacer layer 5.

The first diffusion rate-limiting section 11, the second diffusion rate-limiting section 13, and the third diffusion rate-limiting section 30 are each provided as two horizontally elongated slits, with openings oriented along the longitudinal direction perpendicular to the plane of the drawing. The fourth diffusion rate-limiting section 60 is provided as a single horizontally elongated slit, with openings oriented along the longitudinal direction perpendicular to the plane of the drawing, formed as a gap with the lower surface of the second solid electrolyte layer 6. The area extending from the gas inlet 10 to the third internal cavity 61 is also referred to as the measurement gas flow path.

The sensor element 101 (element body 102) includes a reference gas introduction portion 49, which introduces a reference gas from outside of the sensor element 101 to a reference electrode 42 when measuring the concentrations of the specific gases. The reference gas introduction portion 49 comprises a reference gas introduction space 43 and a reference gas introduction layer 48. The reference gas introduction space 43 is an inward space formed from the rear end surface of the sensor element 101. The reference gas introduction space 43 is located between the upper surface of the third substrate layer 3 and the lower surface of the spacer layer 5, and is laterally defined by the side surfaces of the first solid electrolyte layer 4. The reference gas introduction space 43 opens to the rear end surface of the sensor element 101, with this opening serving as an inlet portion 49a of the reference gas introduction portion 49. The reference gas is introduced into the reference gas introduction space 43 through the inlet portion 49a. The reference gas introduction portion 49 introduces the reference gas, which has entered through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina or the like. A portion of the upper surface of the reference gas introduction layer 48 is exposed within the reference gas introduction space 43. The reference gas introduction layer 48 is formed so as to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4, and as described above, the reference gas introduction layer 48, which is connected to the reference gas introduction space 43, is provided around the reference electrode 42. Furthermore, as will be explained later, the reference electrode 42 enables the measurement of the oxygen concentration (oxygen partial pressure) in the first internal cavity 20, the second internal cavity 40, and the third internal cavity 61. The reference electrode 42 is formed as a porous cermet electrode (for example, a cermet electrode of Pt and ZrO₂).

In the measurement gas flow path, the gas inlet 10 is a portion that is open to the external space, allowing the measurement gas to be drawn into the sensor element 101 from the external space. The first diffusion rate-limiting section 11 is a part that imparts a predetermined diffusion resistance to the measurement gas introduced through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. When the measurement gas is introduced from outside the sensor element 101 into the first internal cavity 20, the measurement gas that is abruptly drawn into the sensor element 101 through the gas inlet 10 due to pressure fluctuations in the external space (such as exhaust pulsations in the case where the measurement gas is automobile exhaust gas) is not directly introduced into the first internal cavity 20. Instead, after the pressure fluctuations of the measurement gas are attenuated through the first diffusion rate-limiting section 11, the buffer space 12, and the second diffusion rate-limiting section 13, the measurement gas is introduced into the first internal cavity 20. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of a main pump cell 21.

The main pump cell 21 is an electrochemical pump cell, which is constituted by an inner pump electrode 22 with a ceiling electrode portion 22a provided on nearly the entire lower surface of the second solid electrolyte layer 6 facing the first internal cavity 20, an outer pump electrode 23, which is provided in a manner exposed to the outside of the sensor element 101 in a region of the upper surface of the second solid electrolyte layer 6 corresponding to the ceiling electrode portion 22a, and the second solid electrolyte layer 6, the spacer layer 5, and the first solid electrolyte layer 4, which form the current path between these electrodes.

The inner pump electrode 22 is formed so as to extend across the upper and lower solid electrolyte layers, (namely the second solid electrolyte layer 6 and the first solid electrolyte layer 4,) and the spacer layer 5 that provides sidewalls, which together define the first internal cavity 20. Specifically, the ceiling electrode portion 22a is formed on the lower surface of the second solid electrolyte layer 6, which constitutes the ceiling surface of the first internal cavity 20, and a bottom electrode portion 22b is formed on the upper surface of the first solid electrolyte layer 4, which constitutes the bottom surface of the first internal cavity 20. Further, in order to connect the ceiling electrode portion 22a and the bottom electrode portion 22b, side electrode portions (not shown) are formed on the side wall surfaces (inner surfaces) of the spacer layer 5, which constitute both sidewall portions of the first internal cavity 20. The inner pump electrode 22 is disposed in a tunnel-like structure at the region where the side electrode portion is provided.

In the main pump cell 21, a desired voltage Vp0 is applied between the inner pump electrode 22 and the outer pump electrode 23, whereby a pump current Ip0 is caused to flow in a positive direction or a negative direction between the inner pump electrode 22 and the outer pump electrode 23. Thus, the oxygen in the first internal cavity 20 can be pumped out to the external space, or the oxygen in the external space can be pumped into the first internal cavity 20.

Further, in order to detect the oxygen concentration (oxygen partial pressure) in the atmosphere within the first internal cavity 20, an electrochemical sensor cell, that is, a main-pump-control oxygen-partial-pressure detection sensor cell 80, is constituted by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, by feedback-controlling the voltage Vp0 of the variable power source 24 such that the voltage V0 reaches a target value, the pump current Ip0 is controlled, thereby adjusting the oxygen concentration in the first internal cavity 20.

The third diffusion rate-limiting section 30 is a part that imparts a predetermined diffusion resistance to the measurement gas, whose oxygen concentration (oxygen partial pressure) is controlled by the operation of the main pump cell 21 in the first internal cavity 20, and guides the measurement gas into the second internal cavity 40.

The second internal cavity 40 is provided as a space in which the oxygen partial pressure is adjusted by the first measurement pump cell 50 for the measurement gas introduced through the third diffusion rate-limiting section 30, to carry out processing for measuring the water concentration in the measurement gas.

The first measurement pump cell 50 is an electrochemical pump cell, which is constituted by a first measurement electrode 51 with a ceiling electrode portion 51a provided on nearly the entire lower surface of the second solid electrolyte layer 6 facing the second internal cavity 40, the outer pump electrode 23 (not limited to the outer pump electrode 23, but may be any suitable electrode disposed on an outer surface of the sensor element 101), the second solid electrolyte layer 6, the spacer layer 5, and the first solid electrolyte layer 4.

The first measurement electrode 51 is disposed within the second internal cavity 40 in a tunnel-like structure similar to that of the inner pump electrode 22 disposed in the first internal cavity 20 described above. Specifically, the ceiling electrode portion 51a is formed on the second solid electrolyte layer 6, which constitutes the ceiling surface of the second internal cavity 40, and a bottom electrode portion 51b is formed on the first solid electrolyte layer 4, which constitutes the bottom surface of the second internal cavity 40. Further, side electrode portions (not shown), which connect the ceiling electrode portion 51a and the bottom electrode portion 51b, are formed on the inner side surfaces of the spacer layer 5, which constitute both sidewall portions of the second internal cavity 40. Thus, the first measurement electrode 51 is formed in a tunnel-like structure.

In the first measurement pump cell 50, a desired voltage Vp1 is applied between the first measurement electrode 51 and the outer pump electrode 23. Thus, the oxygen in the atmosphere within the second internal cavity 40 can be pumped out to the external space, or the oxygen can be pumped into the second internal cavity 40 from the external space.

Further, in order to control the oxygen partial pressure in the atmosphere within the second internal cavity 40, an electrochemical sensor cell, that is, an first-measurement-pump-control oxygen-partial-pressure detection sensor cell 81, is constituted by the first measurement electrode 51, the reference electrode 42, the second solid electrolyte layer 6, the spacer layer 5, the first solid electrolyte layer 4, and the third substrate layer 3.

The first measurement pump cell 50 performs pumping via the variable power source 52, which is voltage-controlled based on an electromotive force (voltage V1) detected by the first-measurement-pump-control oxygen-partial-pressure detection sensor cell 81. As a result, the oxygen partial pressure in the atmosphere of the second internal cavity 40 is adjusted by the pump current Ip1 flowing through the first measurement pump cell 50.

The fourth diffusion rate-limiting section 60 is a part that imparts a predetermined diffusion resistance to the measurement gas, whose oxygen concentration (oxygen partial pressure) is controlled by the operation of the first measurement pump cell 50 in the second internal cavity 40, and guides the measurement gas into the third internal cavity 61.

The third internal cavity 61 is provided as a space in which the oxygen partial pressure is adjusted by the second measurement pump cell 41 for the measurement gas introduced through the fourth diffusion rate-limiting section 60, to carry out processing for measuring the carbon dioxide concentration in the measurement gas.

The second measurement pump cell 41 is an electrochemical pump cell, which is constituted by a second measurement electrode 44 provided on the upper surface of the first solid electrolyte layer 4 facing the third internal cavity 61, the outer pump electrode 23 (not limited to the outer pump electrode 23, but may be any suitable electrode disposed on an outer surface of the sensor element 101), the second solid electrolyte layer 6, the spacer layer 5, and the first solid electrolyte layer 4.

In the second measurement pump cell 41, a desired voltage Vp2 is applied between the second measurement electrode 44 and the outer pump electrode 23. Thus, the oxygen in the atmosphere within the third internal cavity 61 can be pumped out to the external space, or the oxygen can be pumped into the second internal cavity 40 from the external space.

Further, in order to detect the oxygen partial pressure around the second measurement electrode 44, an electrochemical sensor cell, that is, a second-measurement-pump-control oxygen-partial-pressure detection sensor cell 82, is formed by the first solid electrolyte layer 4, the third substrate layer 3, the second measurement electrode 44, and the reference electrode 42.

The variable power source 46 is controlled based on the electromotive force (voltage V2) detected by the second-measurement-pump-control oxygen-partial-pressure detection sensor cell 82, and the voltage Vp2 of the variable power source 46 is applied to the second measurement pump cell 41. As a result, the oxygen partial pressure in the atmosphere of the third internal cavity 61 is adjusted by the pump current Ip2 flowing through the second measurement pump cell 41.

Furthermore, an electrochemical sensor cell 83 is formed from the second solid electrolyte layer 6, the spacer layer 5, the first solid electrolyte layer 4, the third substrate layer 3, the outer pump electrode 23, and the reference electrode 42, and the oxygen partial pressure in the measurement gas outside the sensor can be detected based on the electromotive force (voltage Vref) obtained by this sensor cell 83.

Each of the electrodes 22, 23, 42, 44, and 51 will be described. The inner pump electrode 22, the first measurement electrode 51, and the second measurement electrode 44 each contain a first type of noble metal with catalytic activity. Examples of the first type of noble metal include, but are not limited to, at least one selected from Pt, Rh, Ir, Ru, and Pd. The outer pump electrode 23 and the reference electrode 42 also contain the first type of noble metal. It is preferable that the first measurement electrode 51 further contain a second type of noble metal for suppressing the catalytic activity of the first type of noble metal with respect to carbon monoxide. By containing the second type of noble metal, the oxidation capability of the first measurement electrode 51 with respect to carbon monoxide is reduced. An example of the second type of noble metal is Au. The inner pump electrode 22 and the second measurement electrode 44 do not contain the second type of noble metal. It is preferable that the outer pump electrode 23 and the reference electrode 42 also do not contain the second type of noble metal. Each of the electrodes 22, 23, 42, 44, and 51 is preferably a cermet containing a noble metal and a solid electrolyte having oxygen ion conductivity (e.g. ZrO₂). Each of the electrodes 22, 23, 42, 44, and 51 is also preferably a porous body. In the present embodiment, the first measurement electrode 51 is a porous cermet electrode composed of Pt containing 1% Au and ZrO₂. Additionally, the inner pump electrode 22, the outer pump electrode 23, the reference electrode 42, and the second measurement electrode 44 are each porous cermet electrodes composed of Pt and ZrO₂.

The sensor element 101 includes a heater section 70 that performs temperature regulation by heating and maintaining the temperature of the sensor element 101, in order to enhance the oxygen-ion-conductivity of the solid electrolyte. The heater section 70 includes a heater connector electrode 71, a heater 72, a through hole 73, a heater insulating layer 74, and a pressure relief hole 75.

The heater connector electrode 71 is an electrode formed in such a manner as to be in contact with the lower surface of the first substrate layer 1. By connecting the heater connector electrode 71 to a heater power source 76 (see FIG. 2), power can be supplied from the heater power source 76 to the heater section 70.

The heater 72 is an electrical resistor formed in such a manner as to be sandwiched between the second substrate layer 2 and the third substrate layer 3 from above and below. The heater 72 is connected to the heater connector electrode 71 via the through hole 73, and generates heat when power is supplied from the heater power source 76 through the heater connector electrode 71, thereby heating and maintaining the temperature of the solid electrolyte forming the sensor element 101.

The heater 72 is also embedded across the entire region from the first internal cavity 20 to the third internal cavity 61, making it possible to adjust the temperature of the entire sensor element 101 to a level at which the solid electrolyte is activated.

The heater insulating layer 74 is an insulating layer formed of an insulator such as alumina and provided on the upper and lower surfaces of the heater 72. The heater insulating layer 74 is formed for the purpose of providing electrical insulation between the second substrate layer 2 and the heater 72, as well as between the third substrate layer 3 and the heater 72.

The pressure relief hole 75 is a portion that penetrates through the third substrate layer 3 and the reference gas introduction layer 48, and is formed so as to communicate with the reference gas introduction space 43. The pressure relief hole 75 is formed for the purpose of relieving an increase in internal pressure caused by a rise in temperature within the heater insulating layer 74.

The porous protective layer 90 is a porous body that covers a predetermined region around the front end of the outer surface of the element body 102 (a region of a predetermined range from the front end of the element body 102 toward the rear end side). More specifically, the porous protective layer 90 covers respective portions of the upper, lower, left, and right surfaces of the element body 102 and the entirety of the front end surface. The porous protective layer 90 covers the gas inlet 10 and the outer pump electrode 23. The rear end of the porous protective layer 90 is located rearward of the rear end of the measurement gas flow path. By these arrangements, the porous protective layer 90 covers most of the portion on the front-end side of the element body 102 that is exposed to the measurement gas, thereby protecting that portion. The porous protective layer 90, for example, serves to suppress cracking of the element body 102 caused by adhesion of moisture in the measurement gas. Although the porous protective layer 90 covers the gas inlet 10, since the porous protective layer 90 is a porous body, the measurement gas can flow through the interior of the porous protective layer 90 and reach the gas inlet 10.

The porous protective layer 90 is made of a ceramic porous body such as an alumina porous body, a zirconia porous body, a spinel porous body, a cordierite porous body, a titania porous body, or a magnesia porous body. In this embodiment, the porous protective layer 90 is made of an alumina porous body. The porosity of the porous protective layer 90 may be, for example, 10% or more and 70% or less. The thickness (length in the front-rear direction) of the portion of the porous protective layer 90 that covers the gas inlet 10 may be, for example, 850 μm or less.

As shown in FIG. 2, the control device 95 includes the variable power sources 24, 46, and 52 described above, the heater power source 76 also described above, and a control unit 96. The control unit 96 is a microprocessor including a CPU 97 and a storage unit 98. The storage unit 98 is a rewritable non-volatile memory, and is capable of storing, for example, various programs and various kinds of data. The control unit 96 inputs the voltage V0 from the main-pump-control oxygen-partial-pressure detection sensor cell 80, the voltage V1 from the first-measurement-pump-control oxygen-partial-pressure detection sensor cell 81, the voltage V2 from the second-measurement-pump-control oxygen-partial-pressure detection sensor cell 82, the voltage Vref from the sensor cell 83, the pump current Ip0 flowing through the main pump cell 21, the pump current Ip1 flowing through the first measurement pump cell 50, and the pump current Ip2 flowing through the second measurement pump cell 41. In addition, the control unit 96 controls the voltages Vp0, Vp1, and Vp2 output from the variable power sources 24, 52, and 46, respectively, by outputting control signals to the variable power sources 24, 52, and 46. Through this control, the main pump cell 21, the first measurement pump cell 50, and the second measurement pump cell 41 are controlled. The control unit 96 also controls the power supplied from the heater power source 76 to the heater 72 by outputting a control signal to the heater power source 76. The storage unit 98 also stores target values V0*, V1*, and V2*, which will be described later. The CPU 97 of the control unit 96 performs control of the respective cells 21, 50, and 41 with reference to the target values V0*, V1*, and V2*.

The control unit 96 performs a main pump control processing (an example of the first pump cell control processing), which controls the main pump cell 21 to pump out oxygen from around the inner pump electrode 22 to around the outer pump electrode 23. Specifically, the control unit 96 feedback-controls the voltage Vp0 of the variable power source 24 so that the voltage V0 reaches a target value V0*, thereby controlling the main pump cell 21. The target value V0* is set as a value such that the oxygen concentration in the first internal cavity 20 reaches a predetermined low concentration that is sufficiently low to substantially reduce all of the water and carbon dioxide in the measurement gas. By performing this main pump control processing, in the first internal cavity 20, water in the measurement gas is reduced to generate hydrogen and oxygen, and carbon dioxide in the measurement gas is reduced to generate carbon monoxide and oxygen. The generated oxygen is pumped out from around the inner pump electrode 22 to around the outer pump electrode 23 by the pump current Ip0 flowing through the main pump cell 21.

The control unit 96 performs a first measurement pump control processing (an example of the second pump cell control processing), which controls the first measurement pump cell 50 to pump into oxygen from around the outer pump electrode 23 to around the first measurement electrode 51. Specifically, the control unit 96 feedback-controls the voltage Vp1 of the variable power source 52 so that the voltage V1 reaches the target value V1*, thereby controlling the first measurement pump cell 50. The target value V1* is set as a value such that the oxygen concentration in the second internal cavity 40 reaches a predetermined concentration sufficient to substantially oxidize all of hydrogen in the second internal cavity 40. By performing this first measurement pump control processing, in the second internal cavity 40, hydrogen generated by the reduction of water in the first internal cavity 20 is oxidized to generate water again. At this time, the pump current Ip1 flowing through the first measurement pump cell 50 correlates with the amount of oxygen pumped into the second internal cavity 40 to oxidize the hydrogen in the second internal cavity 40, and hence correlates with the amount of water in the measurement gas in the first internal cavity 20, which is the source of the hydrogen in the second internal cavity 40. Therefore, the pump current Ip1 correlates with the water concentration in the measurement gas.

Accordingly, the control unit 96 performs a water concentration measurement processing for measuring the water concentration in the measurement gas based on the pump current Ip1 flowing through the first measurement pump cell 50 by the first measurement pump control processing. For example, the control unit 96 derives the water concentration using a water concentration derivation map. The water concentration derivation map, which defines a correspondence between the absolute value of the pump current Ip1 and the water concentration, is predetermined by experiments or analyses and stored in the storage unit 98. In the water concentration measurement processing, the control unit 96 applies the measured absolute value of the pump current Ip1 to the water concentration derivation map to derive the water concentration corresponding to the absolute value of the pump current Ip1. In this way, the water concentration in the measurement gas is measured. Instead of the water concentration derivation map, a relational expression (for example, a linear or quadratic expression) representing the correspondence between the pump current Ip1 and the water concentration may be stored in the storage unit 98.

The control unit 96 performs a second measurement pump control processing (an example of the third pump cell control processing), which controls the second measurement pump cell 41 to pump into oxygen from around the outer pump electrode 23 to around the second measurement electrode 44. Specifically, the control unit 96 feedback-controls the voltage Vp2 of the variable power source 46 so that the voltage V2 reaches the target value V2*, thereby controlling the second measurement pump cell 41. The target value V2* is set as a value such that the oxygen concentration in the third internal cavity 61 reaches a predetermined concentration sufficient to substantially oxidize all of carbon monoxide in the third internal cavity 61. By performing this second measurement pump control processing, in the third internal cavity 61, carbon monoxide generated by the reduction of carbon dioxide in the first internal cavity 20 is oxidized to generate carbon dioxide again. At this time, the pump current Ip2 flowing through the second measurement pump cell 41 correlates with the amount of oxygen pumped into the third internal cavity 61 to oxidize the carbon monoxide in the third internal cavity 61, and hence correlates with the amount of carbon dioxide in the measurement gas in the first internal cavity 20, which is the source of the carbon monoxide in the third internal cavity 61. Therefore, the pump current Ip2 correlates with the carbon dioxide concentration in the measurement gas.

Accordingly, the control unit 96 performs a carbon dioxide concentration measurement processing for measuring the carbon dioxide concentration in the measurement gas based on the pump current Ip2 flowing through the second measurement pump cell 41 by the second measurement pump control processing. For example, the control unit 96 derives the carbon dioxide concentration using a carbon dioxide concentration derivation map. The carbon dioxide concentration derivation map, which defines a correspondence between the absolute value of the pump current Ip2 and the carbon dioxide concentration, is predetermined by experiments or analyses and stored in the storage unit 98. In the carbon dioxide concentration measurement processing, the control unit 96 applies the measured absolute value of the pump current Ip2 to the carbon dioxide concentration derivation map to derive the carbon dioxide concentration corresponding to the absolute value of the pump current Ip2. In this way, the carbon dioxide concentration in the measurement gas is measured. Instead of the carbon dioxide concentration derivation map, a relational expression (for example, a linear or quadratic expression) representing the correspondence between the pump current Ip2 and the carbon dioxide concentration may be stored in the storage unit 98.

Both hydrogen and carbon monoxide generated in the first internal cavity 20 reach the second internal cavity 40. However, hydrogen has a faster gas diffusion rate than carbon monoxide and hydrogen binds with oxygen more readily than carbon monoxide does. Therefore, in the second internal cavity 40, hydrogen can be selectively oxidized, among hydrogen and carbon monoxide, by the first measurement pump control processing. Additionally, since hydrogen rarely reaches the third internal cavity 61 downstream of the second internal cavity 40, the second measurement pump control processing can oxidize carbon monoxide. Further, in this embodiment, as described above, the first measurement electrode 51 has its oxidation capability with respect to carbon monoxide reduced by containing the second type of noble metal. Therefore, in a vicinity of the first measurement electrode 51, that is, in the second internal cavity 40, hydrogen can be more selectively oxidized than carbon monoxide by the first measurement pump control processing.

The control unit 96 performs heater control processing by outputting a control signal to the heater power source 76 such that the temperature of the heater 72 reaches to a target temperature (e.g. 800° C.). Here, the target temperature of the heater 72 is defined as a temperature obtained by adding a margin to the temperature at which the above-described solid electrolyte is activated. The temperature of the heater 72 can be expressed as a linear function of the resistance value of the heater 72. In the heater control processing, the control unit 96 calculates the resistance value of the heater 72, which is a value can be regarded as the temperature of the heater 72 (a value that can be converted into temperature), and feedback-controls the heater power source 76 such that the calculated resistance value reaches to a target resistance value (a value corresponding to the target temperature). The control unit 96, for example, can acquire the voltage of the heater 72 and the current flowing through the heater 72, then calculate the resistance of the heater 72 based on the acquired voltage and current. The control unit 96 may use a three-wire or four-wire method to calculate the resistance, for example. The heater power source 76 adjusts the power supplied to the heater 72 by changing the voltage applied to the heater 72 based on a control signal from the control unit 96 when energizing the heater 72.

In addition, the control device 95, which includes the variable power sources 24, 46, 52, and the heater power source 76 shown in FIG. 2, is actually connected to the electrodes inside the sensor element 101 through unillustrated lead wires formed within the sensor element 101 and unillustrated connector electrodes formed at the rear end of the sensor element 101 (only the heater connector electrode 71 shown in FIG. 1).

Next, a method for manufacturing the gas sensor 100 will be described. In the manufacturing method of the gas sensor 100, the element body 102 is first manufactured, and then a porous protective layer 90 is formed on the element body 102 to manufacture the sensor element 101.

First, a method for manufacturing the element body 102 will be described. Six unfired ceramic green sheets are prepared. In these green sheets, a plurality of sheet holes for positioning during printing and lamination and necessary through holes, etc., are formed in advance. In addition, in the green sheet that becomes the spacer layer 5, a space that will become the measurement gas flow path is provided in advance by a punching process or the like. In the green sheet that becomes the first solid electrolyte layer 4, a space that will become the reference gas introduction space 43 is provided in advance by a punching process or the like. Then, pattern printing processing for forming various patterns is performed on each ceramic green sheet corresponding to the first substrate layer 1, the second substrate layer 2, the third substrate layer 3, the first solid electrolyte layer 4, the spacer layer 5, and the second solid electrolyte layer 6. The patterns to be formed include, specifically, for example, the respective electrodes such as the above-described inner pump electrode 22, the lead wires connected to the respective electrodes, the reference gas introduction layer 48, the heater connector electrode 71, and the heater section 70. Pattern printing is performed by applying, onto the green sheet using a known screen-printing technique, a pattern-forming paste prepared according to the properties required for each formation target. After the pattern printing processing, a drying process is carried out using a known drying means. After the pattern printing and drying are completed, printing and drying of an adhesive paste for laminating and bonding the green sheets corresponding to the respective layers are performed. Then, a pressure bonding process is performed in which the green sheets on which the adhesive paste has been formed are positioned by the sheet holes, laminated in a predetermined order, and bonded by applying predetermined temperature and pressure conditions to be pressure bonded into a single laminated body. In portions of the laminated body that will become spaces such as the measurement gas flow path, a vanishing material (for example, an organic material such as theobromine) that vanishes during firing may be filled. The obtained laminated body contains a plurality of element bodies 102. The laminated body is then cut to the size of the element body 102. The cut laminated body is fired at a predetermined firing temperature to obtain the element body 102.

The first to third diffusion rate-limiting sections 11, 13, and 30 can be formed by applying the above-described vanishing material to the upper and lower surfaces of the green sheet that becomes the spacer layer 5 during the pattern printing processing, and then vanishing the vanishing material during firing. Similarly, the fourth diffusion rate-limiting section 60 can be formed by applying the vanishing material to the upper surface of the green sheet that becomes the spacer layer 5 during the pattern printing processing, and then vanishing the vanishing material during firing. By adjusting the thickness of the vanishing material, the diffusion resistance of each of the first to fourth diffusion rate-limiting sections 11, 13, 30, and 60 can be adjusted.

Next, a porous protective layer 90 is formed on the element body 102 by plasma spraying. For example, using a plasma gun (not shown), a powder spraying material is heated/melted and accelerated by plasma and made to collide with the surface of the element body 102, and the powder spraying material is rapidly solidified to form the porous protective layer 90. The powder spraying material contains a raw powder serving as the material of the above-described porous protective layer 90; in this embodiment, alumina powder is used as the raw powder. When the porous protective layer 90 is formed by plasma spraying, plasma spraying may be performed separately for each of a plurality of faces of the element body 102 on which the porous protective layer 90 is to be formed (here, the upper face, lower face, left face, right face, and front end face). Note that the formation of the porous protective layer 90 is not limited to plasma spraying and can be carried out by various methods such as mold casting, screen printing, and dipping.

By forming the porous protective layer 90 on the element body 102 as described above, the sensor element 101 is obtained. Thereafter, a gas sensor 100 incorporating the sensor element 101 is manufactured. For example, an element sealing unit is attached to and sealingly fixes the sensor element 101, and a connector and lead wires are attached to the rear-end side of the sensor element 101. A protective cover is attached to the front-end side of the sensor element 101 among the element sealing unit. In addition, a outer cylinder is attached to the rear-end side of the sensor element 101 among the element sealing unit, and lead wires are drawn to the outside from the outer cylinder. Then, by connecting the drawn lead wires to the control device 95, the respective electrodes of the sensor element 101 and the heater 72 are electrically connected to the control device 95. It should be noted that the process of assembling the gas sensor 100 by incorporating such a sensor element 101 is known, and is described, for example, in Japanese Unexamined Patent Application Publication No. 2015-178988.

A usage example of the gas sensor 100 constructed as above will be described below. The CPU 97 of the control unit 96 first performs the heater control processing described above to control the heater 72 such that its temperature reaches the target temperature. When the temperature of the heater 72 reaches the target temperature (or near the target temperature), the CPU 97 starts the control of each pump cell 21, 50, and 41 (main pump control processing, first measurement pump control processing, and second measurement pump control processing) described above, and starts acquiring each of the voltages V0, V1, V2, and Vref from each of the sensor cells 80 to 83 described above. While continuously performing these processes, the CPU 97 of the control unit 96 performs the above-described water concentration measurement processing and carbon dioxide concentration measurement processing to measure the water concentration and the carbon dioxide concentration in the measurement gas.

The present inventors investigated the relationship between the responsiveness of the pump currents Ip0, Ip1, and Ip2 to changes in the concentration of gases in the measurement gas and the diffusion resistance of the measurement gas flow path. Since it is difficult to directly measure the diffusion resistance of the measurement gas flow path in an actual sensor element 101, a limiting current was used as a value correlated with the diffusion resistance. Specifically, as a value having a negative correlative relationship with the diffusion resistance from the outside of the sensor element 101 to the first internal cavity 20 of the measurement gas flow path, the limiting current Ir [mA] of the pump current Ip0 was used. As a value having a negative correlative relationship with the diffusion resistance from the outside of the sensor element 101 to the second internal cavity 40 of the measurement gas flow path, the limiting current Ih [mA] of the pump current Ip1 was used. As a value having a negative correlative relationship with the diffusion resistance from the outside of the sensor element 101 to the third internal cavity 61 of the measurement gas flow path, the limiting current Ic [mA] of the pump current Ip2 was used.

The limiting current Ir was taken as the value measured as follows. First, as the measurement gas, a model gas with nitrogen as a base gas and an oxygen concentration of 20% was prepared, and the front-end periphery including the gas inlet 10 of the sensor element 101 was exposed to the atmosphere of this model gas. In addition, the control unit 96 executed the above-described heater control processing to bring the temperature of the heater 72 to around the target temperature. In this state, while gradually increasing the setting value of the target value V0*, the control unit 96 executed the same processing as the above-described main pump control processing. As the target value V0* is gradually increased, the pump current Ip0 increases accordingly; however, when the target value V0* is further increased, a state is reached in which the pump current Ip0 no longer increases even if the target value V0* changes. That is, the pump current Ip0 reaches the limiting current. This limiting current value of the pump current Ip0 was taken as the limiting current Ir. In other words, the limiting current Ir is the limiting current when the main pump cell 21 pumps oxygen from around the inner pump electrode 22 to around the outer pump electrode 23, with the front-end periphery of the sensor element 101 exposed to the atmosphere of a model gas having nitrogen as a base gas and an oxygen concentration of 20%. The limiting currents Ih and Ic were measured in the same manner. The limiting current Ih is the limiting current when the first measurement pump cell 50 pumps oxygen from around the first measurement electrode 51 to around the outer pump electrode 23 with the front-end periphery of the sensor element 101 exposed to the atmosphere of a model gas having nitrogen as a base gas and an oxygen concentration of 20%. The limiting current Ic is the limiting current when the second measurement pump cell 41 pumps oxygen from around the second measurement electrode 44 to around the outer pump electrode 23 with the front-end periphery of the sensor element 101 exposed to the atmosphere of a model gas having nitrogen as a base gas and an oxygen concentration of 20%. Note that, when measuring the limiting current Ir, the first and second measurement pump cells 50 and 41 were not operated. When measuring the limiting current Ih, the main pump cell 21 and the second measurement pump cell 41 were not operated. When measuring the limiting current Ic, the main pump cell 21 and the first measurement pump cell 50 were not operated. As described above, the limiting currents Ir, Ih, and Ic are values having a negative correlative relationship with the diffusion resistance of the measurement gas flow path. That is, the limiting currents Ir, Ih, and Ic are values representing structural characteristics of the measurement gas flow path of the sensor element 101.

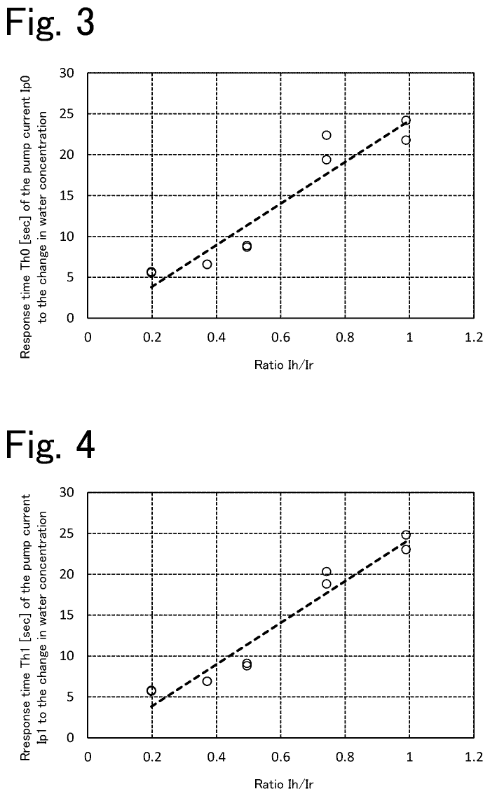

The present inventors investigated, as follows, the relationship among the responsiveness of the pump currents Ip0 and Ip1 to changes in the water concentration in the measurement gas and the ratio Ih/Ir of the limiting current Ih to the limiting current Ir as a value representing the diffusion resistance of the measurement gas flow path. First, sensor elements 101 having the same structure except that the diffusion resistance of the third diffusion rate-limiting section 30 was made different so that the limiting current Ih differed from one another were fabricated as Experimental Examples 1 to 10. The diffusion resistance of the third diffusion rate-limiting section 30 was adjusted by changing the height of the third diffusion rate-limiting section 30 (two slits) by changing the thickness of the vanishing material applied to the portions corresponding to the third diffusion rate-limiting section 30 on the upper and lower surfaces of the green sheet that becomes the spacer layer 5 in the above-described manufacturing method of the sensor element 101. Next, as the measurement gas, a model gas having nitrogen as a base gas, an oxygen concentration of 0%, a water concentration of 1%, and a carbon dioxide concentration of 0% was prepared, and the front-end periphery of the sensor element 101 of Experimental Example 1 was exposed to the atmosphere of this model gas. In addition, the control unit 96 executed the above-described heater control processing to bring the temperature of the heater 72 to around the target temperature. In this state, the control device 95 executed the main pump control processing, the first measurement-pump control processing, and the second measurement-pump control processing described above, and waited until the pump currents Ip0, Ip1, and Ip2 stabilized. Subsequently, the water concentration of the model gas was abruptly changed from 1% to 5%, and after the pump currents Ip0 and Ip1 rose sharply, it was waited until they stabilized again. For each of the pump currents Ip0, Ip1, and Ip2, stabilization was determined when the time variation of the current value became 0.2%/see or less. Then, taking the value of the pump current Ip0 immediately before the abrupt change in water concentration as 0% and the value when the pump current Ip0 stabilized again after the abrupt change in water concentration (the average value over 10 seconds after stabilization) as 100%, the time from the abrupt change in water concentration until the pump current Ip0 reached 90% was measured as the response time Th0 [sec] of the pump current Ip0 to the change in water concentration. Similarly, taking the value of the pump current Ip1 immediately before the abrupt change in water concentration as 0% and the value when the pump current Ip1 stabilized again after the abrupt change as 100%, the time from the abrupt change in water concentration until the pump current Ip1 reached 90% was measured as the response time Th1 [sec] of the pump current Ip1 to the change in water concentration. For the sensor elements 101 of Experimental Examples 2 to 10 as well, the response times Th0 and Th1 were measured in the same manner. The shorter the response time Th0, the higher the responsiveness of the pump current Ip0 to changes in the water concentration. The shorter the response time Th1, the higher the responsiveness of the pump current Ip1 to changes in the water concentration, and hence the higher the responsiveness of the measurement of the water concentration (the measured value of the water concentration in the water concentration measurement processing) to changes in the water concentration. The ratios Ih/Ir, the response times Th0, and the response times Th1 for each of Experimental Examples 1 to 10 are shown in Table 1 and FIGS. 3 and 4. In FIGS. 3 and 4, approximation straight lines calculated from the results of Experimental Examples 1 to 10 are shown by broken lines.

TABLE 1

| | | Response | Response |
|---|---|---|---|
| | Ratio | time Th0 | time Th1 |
| | Ih/Ir | [sec] of Ip0 | [sec] of Ip1 |
| Experimental Example 1 | 0.98972 | 21.8 | 24.8 |
| Experimental Example 2 | 0.98972 | 24.2 | 23.0 |
| Experimental Example 3 | 0.74229 | 22.4 | 18.8 |
| Experimental Example 4 | 0.74229 | 19.4 | 20.3 |
| Experimental Example 5 | 0.49486 | 8.7 | 9.1 |
| Experimental Example 6 | 0.49486 | 8.9 | 8.8 |
| Experimental Example 7 | 0.37115 | 6.6 | 6.9 |
| Experimental Example 8 | 0.37115 | 6.6 | 6.9 |
| Experimental Example 9 | 0.19794 | 5.6 | 5.7 |
| Experimental Example 10 | 0.19794 | 5.7 | 5.8 |

As shown in Table 1 and FIGS. 3 and 4, it was confirmed that the smaller the ratio Ih/Ir, the smaller the response times Th0 and Th1 tend to be. In Experimental Examples 5 to 10, the response time Th1 was 10 seconds or less, indicating high responsiveness in measuring the water concentration. In addition, the approximation straight line shown in FIG. 4 was the following expression (1). From this expression (1), it was confirmed that when the ratio Ih/Ir is 0.44 or less, the response time Th1 becomes 10 seconds or less, and the responsiveness of the measurement of the water concentration can be enhanced. In the sensor element 101 of this embodiment, the flow path cross-sectional areas and front-rear lengths of the first diffusion rate-limiting section 11, the second diffusion rate-limiting section 13, and the third diffusion rate-limiting section 30 are adjusted so that this ratio Ih/Ir becomes 0.44 or less, thereby enhancing the responsiveness of the measurement of the water concentration.

Th1=25.506×(Ih/Ir)−1.2527 (1)

The reason why the responsiveness of the measurement of the water concentration can be enhanced when the ratio Ih/Ir is 0.44 or less is considered as follows. First, as described above, the limiting current Ir has the negative correlative relationship with the diffusion resistance from the outside of the sensor element 101 to the first internal cavity 20 of the measurement gas flow path. The limiting current Ih has the negative correlative relationship with the diffusion resistance from the outside of the sensor element 101 to the second internal cavity 40 of the measurement gas flow path. Therefore, the larger the ratio Ih/Ir, the smaller the proportion of water that flows into the first internal cavity 20 from the outside among the water in the measurement gas in the first internal cavity 20, and the larger the proportion of water that back-diffuses from the second internal cavity 40 to the first internal cavity 20. The water that back-diffuses from the second internal cavity 40 to the first internal cavity 20 is explained below. Water in the first internal cavity 20 is reduced by the main pump control processing to generate hydrogen, the hydrogen reaches the second internal cavity 40 and is oxidized by the first measurement-pump control processing to become water again. This water again reaches the first internal cavity 20 by back diffusion. Moreover, the larger the ratio Ih/Ir, the smaller the diffusion resistance from the second internal cavity 40 to the first internal cavity 20 tends to be relative to the diffusion resistance from the outside of the sensor element 101 to the first internal cavity 20 of the measurement gas flow path; therefore, there is a tendency for more water to reach the first internal cavity 20 due to back diffusion. Consequently, the larger the ratio Ih/Ir, the smaller the proportion of water that flows into the first internal cavity 20 from the outside among the water in the measurement gas in the first internal cavity 20, and the larger the proportion of water that back-diffuses from the second internal cavity 40 to the first internal cavity 20, so that a discrepancy is likely to occur between the amount of hydrogen generated by the reduction of water in the first internal cavity 20 by the main pump control processing and the amount of water in the measurement gas outside the sensor element 101. As a result, when the water concentration in the measurement gas outside the sensor element 101 changes, the time until the amount of hydrogen generated by the reduction of water in the first internal cavity 20 corresponds to the changed water concentration becomes longer. That is, it is considered that the larger the ratio Ih/Ir, the lower the responsiveness of the pump current Ip0. In fact, although the sensor elements 101 of Experimental Examples 1 to 10 had the same limiting current Ir—that is, the diffusion resistance from the outside of the sensor element 101 to the first internal cavity 20 was the same—the tendency that the larger the ratio Ih/Ir, the longer the response time Th0, i.e., the lower the responsiveness of the pump current Ip0, was confirmed as shown in Table 1 and FIG. 3. Therefore, in Experimental Examples 1 to 10, the larger the ratio Ih/Ir—that is, the smaller the diffusion resistance between the first internal cavity 20 and the second internal cavity 40—the longer the response time Th0 became, which is considered to be due to the above-described back diffusion of water. When the responsiveness of the pump current Ip0 is low, the time until the pump current Ip1 corresponds to the changed water concentration becomes longer; in other words, the responsiveness of the pump current Ip1 also becomes lower. Therefore, it is considered that the larger the ratio Ih/Ir, the longer the response time Th1 becomes, and such a tendency was actually confirmed in Table 1 and FIG. 4. Accordingly, when the ratio Ih/Ir is 0.44 or less, the proportion of water that back-diffuses from the second internal cavity 40 to the first internal cavity 20 among the water in the measurement gas in the first internal cavity 20 can be made sufficiently small, and it is therefore considered that the responsiveness of the measurement of the water concentration can be improved.

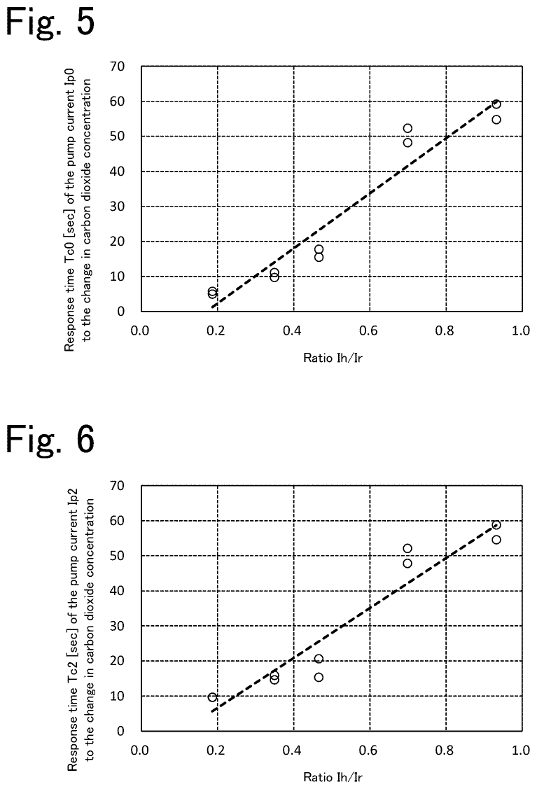

The present inventors investigated, as follows, the relationship among the responsiveness of the pump currents Ip0 and Ip2 to changes in the carbon dioxide concentration in the measurement gas and the ratio Ic/Ir of the limiting current Ic to the limiting current Ir as a value representing the diffusion resistance of the measurement gas flow path. First, sensor elements 101 having the same structure except that the diffusion resistance of the fourth diffusion rate-limiting section 60 was made different so that the limiting current Ic differed from one another were fabricated as Experimental Examples 11 to 20. The diffusion resistance of the fourth diffusion rate-limiting section 60 was adjusted by changing the height of the fourth diffusion rate-limiting section 60 (a single slit) by changing the thickness of the vanishing material applied to the portion corresponding to the fourth diffusion rate-limiting section 60 on the upper surface of the green sheet that becomes the spacer layer 5 in the above-described manufacturing method of the sensor element 101. Next, as the measurement gas, a model gas having nitrogen as a base gas, an oxygen concentration of 0%, a water concentration of 0%, and a carbon dioxide concentration of 1% was prepared, and the front-end periphery of the sensor element 101 of Experimental Example 11 was exposed to the atmosphere of this model gas. In addition, the control unit 96 executed the above-described heater control processing to bring the temperature of the heater 72 to around the target temperature. In this state, the control device 95 executed the above-described main pump control processing, first measurement-pump control processing, and second measurement-pump control processing, and waited until the pump currents Ip0, Ip1, and Ip2 stabilized. Subsequently, the carbon dioxide concentration of the model gas was abruptly changed from 1% to 5%, and after the pump currents Ip0 and Ip2 rose sharply, it was waited until they stabilized again. For each of the pump currents Ip0, Ip1, and Ip2, stabilization was determined when the time variation of the current value became 0.2%/see or less. Then, taking the value of the pump current Ip0 immediately before the abrupt change in carbon dioxide concentration as 0% and the value when the pump current Ip0 stabilized again after the abrupt change (the average value over 10 seconds after stabilization) as 100%, the time from the abrupt change in carbon dioxide concentration until the pump current Ip0 reached 90% was measured as the response time Tc0 [sec] of the pump current Ip0 to the change in carbon dioxide concentration. Similarly, taking the value of the pump current Ip2 immediately before the abrupt change in carbon dioxide concentration as 0% and the value when the pump current Ip2 stabilized again after the abrupt change as 100%, the time from the abrupt change in carbon dioxide concentration until the pump current Ip2 reached 90% was measured as the response time Tc2 [sec] of the pump current Ip2 to the change in carbon dioxide concentration. For the sensor elements 101 of Experimental Examples 12 to 20 as well, the response times Tc0 and Tc2 were measured in the same manner. The shorter the response time Tc0, the higher the responsiveness of the pump current Ip0 to changes in the carbon dioxide concentration. The shorter the response time Tc2, the higher the responsiveness of the pump current Ip2 to changes in the carbon dioxide concentration, and hence the higher the responsiveness of the measurement of the carbon dioxide concentration (the measured value of the carbon dioxide concentration in the carbon dioxide concentration measurement processing) to changes in the carbon dioxide concentration. The ratios Ic/Ir, the response times Tc0, and the response times Tc2 for each of Experimental Examples 11 to 20 are shown in Table 2 and FIGS. 5 and 6. In FIGS. 5 and 6, approximation straight lines calculated from the results of Experimental Examples 11 to 20 are shown by broken lines.

TABLE 2

| | | Response | Response |
|---|---|---|---|
| | Ratio | time Tc0 | time Tc2 |
| | Ic/Ir | [sec] of Ip0 | [sec] of Ip2 |
| Experimental Example 11 | 0.93181 | 54.8 | 54.6 |
| Experimental Example 12 | 0.93181 | 59.2 | 58.8 |
| Experimental Example 13 | 0.69886 | 52.3 | 52.1 |
| Experimental Example 14 | 0.69886 | 48.2 | 47.8 |
| Experimental Example 15 | 0.46591 | 15.5 | 15.3 |
| Experimental Example 16 | 0.46591 | 17.8 | 20.6 |
| Experimental Example 17 | 0.34943 | 11.1 | 15.9 |
| Experimental Example 18 | 0.34943 | 9.7 | 14.6 |
| Experimental Example 19 | 0.18636 | 5.0 | 9.6 |
| Experimental Example 20 | 0.18636 | 5.8 | 9.6 |

As shown in Table 2 and FIGS. 5 and 6, it was confirmed that the smaller the ratio Ic/Ir, the smaller the response times Tc0 and Tc2 tend to be. In Experimental Examples 19 to 20, the response time Tc2 was 10 seconds or less, indicating high responsiveness in measuring the carbon dioxide concentration. In addition, the approximation straight line shown in FIG. 6 was the following expression (2). From this expression (2), it was confirmed that when the ratio Ic/Ir is 0.24 or less, the response time Tc2 becomes 10 seconds or less, and the responsiveness of the measurement of the carbon dioxide concentration can be enhanced. In the sensor element 101 of this embodiment, the flow path cross-sectional areas and front-rear lengths of the first diffusion rate-limiting section 11, the second diffusion rate-limiting section 13, the third diffusion rate-limiting section 30, and the fourth diffusion rate-limiting section 60 are adjusted so that this ratio Ic/Ir becomes 0.24 or less, thereby enhancing the responsiveness of the measurement of the carbon dioxide concentration.

Tc2=71.195×(Ic/Ir)−7.5921 (2)

The reason why the responsiveness of the measurement of the carbon dioxide concentration can be enhanced when the ratio Ic/Ir is 0.24 or less is considered as follows. First, as described above, the limiting current Ir has the negative correlative relationship with the diffusion resistance from the outside of the sensor element 101 to the first internal cavity 20 of the measurement gas flow path. The limiting current Ic has the negative correlative relationship with the diffusion resistance from the outside of the sensor element 101 to the third internal cavity 61 of the measurement gas flow path. Therefore, the larger the ratio Ic/Ir, the smaller the proportion of carbon dioxide that flows into the first internal cavity 20 from the outside among the carbon dioxide in the measurement gas in the first internal cavity 20, and the larger the proportion of carbon dioxide that back-diffuses from the third internal cavity 61 to the first internal cavity 20. The carbon dioxide that back-diffuses from the third internal cavity 61 to the first internal cavity 20 is explained below. Carbon dioxide in the first internal cavity 20 is reduced by the main pump control processing to generate carbon monoxide, the carbon monoxide reaches the third internal cavity 61 and is oxidized by the second measurement-pump control processing to become carbon dioxide again. This carbon dioxide again reaches the first internal cavity 20 by back diffusion. Moreover, the larger the ratio Ic/Ir, the smaller the diffusion resistance from the third internal cavity 61 to the first internal cavity 20 tends to be relative to the diffusion resistance from the outside of the sensor element 101 to the first internal cavity 20 of the measurement gas flow path; therefore, there is a tendency for more carbon dioxide to reach the first internal cavity 20 due to back diffusion. Consequently, the larger the ratio Ic/Ir, the smaller the proportion of carbon dioxide that flows into the first internal cavity 20 from the outside among the carbon dioxide in the measurement gas in the first internal cavity 20, and the larger the proportion of carbon dioxide that back-diffuses from the third internal cavity 61 to the first internal cavity 20, so that a discrepancy is likely to occur between the amount of carbon monoxide generated by the reduction of carbon dioxide in the first internal cavity 20 by the main pump control processing and the amount of carbon dioxide in the measurement gas outside the sensor element 101. As a result, when the carbon dioxide concentration in the measurement gas outside the sensor element 101 changes, the time until the amount of carbon monoxide generated by the reduction of carbon dioxide in the first internal cavity 20 corresponds to the changed carbon dioxide concentration becomes longer. That is, it is considered that the larger the ratio Ic/Ir, the lower the responsiveness of the pump current Ip0. In fact, although the sensor elements 101 of Experimental Examples 11 to 20 had the same limiting current Ir—that is, the diffusion resistance from the outside of the sensor element 101 to the first internal cavity 20 was the same—the tendency that the larger the ratio Ic/Ir, the longer the response time Tc0, i.e., the lower the responsiveness of the pump current Ip0, was confirmed as shown in Table 2 and FIG. 5. Therefore, in Experimental Examples 11 to 20, the larger the ratio Ic/Ir—that is, the smaller the diffusion resistance between the first internal cavity 20 and the third internal cavity 61—the longer the response time Tc0 became, which is considered to be due to the above-described back diffusion of carbon dioxide. When the responsiveness of the pump current Ip0 is low, the time until the pump current Ip2 corresponds to the changed carbon dioxide concentration becomes longer; in other words, the responsiveness of the pump current Ip2 also becomes lower. Therefore, it is considered that the larger the ratio Ic/Ir, the longer the response time Tc2 becomes, and such a tendency was actually confirmed in Table 2 and FIG. 6. Accordingly, when the ratio Ic/Ir is 0.24 or less, the proportion of carbon dioxide that back-diffuses from the third internal cavity 61 to the first internal cavity 20 among the carbon dioxide in the measurement gas in the first internal cavity 20 can be made sufficiently small, and it is therefore considered that the responsiveness of the measurement of the carbon dioxide concentration can be improved.

Note that the upper limits for improving responsiveness differ between the ratio Ih/Ir and the ratio Ic/Ir presumably because the diffusion rates of water (and hydrogen) and carbon dioxide (and carbon monoxide) are different.

Experimental Examples 5 to 10 and 19 to 20 correspond to embodiments of the present invention, while Experimental Examples 1 to 4 and 11 to 18 correspond to comparative examples. However, the present invention is not limited to these embodiments.

Preferably, the limiting current Ih is 0.3 mA or less. If the limiting current Ih is too large, that is, if the diffusion resistance from the outside of the sensor element 101 to the second internal cavity 40 is too small, the flow rate of hydrogen flowing into the second internal cavity 40 may become too large, and the oxidation of hydrogen in the first measurement pump control processing may become insufficient. As a result, the pump current Ip1 and the water concentration may cease to correspond with good accuracy, potentially lowering the measurement accuracy of the water concentration. If the limiting current Ih is 0.3 mA or less, such a decrease in the measurement accuracy of the water concentration can be suppressed. Similarly, it is preferable that the limiting current Ic be 0.3 mA or less. If the limiting current Ic is 0.3 mA or less, it is possible to suppress insufficiency in the oxidation of carbon monoxide in the third internal cavity 61 by the second measurement pump control processing and thereby suppress a decrease in the measurement accuracy of the carbon dioxide concentration.

The limiting current Ih may be 0.05 mA or more. The limiting current Ic may be 0.05 mA or more. The limiting current Ir may be 0.1 mA or more. The limiting current Ir may be 1.5 mA or less.

Here, the correspondence relationship between the elements according to the present embodiment and the elements according to the present invention will be clarified. The sensor element 101 according to the present embodiment corresponds to the sensor element according to the invention. The control device 95 corresponds to the control device. The element body 102 corresponds to the element body. The first internal cavity 20 corresponds to the first chamber. The inner pump electrode 22 corresponds to the first inner electrode. The main pump cell 21 corresponds to the first pump cell. The second internal cavity 40 corresponds to the second chamber. The first measurement electrode 51 corresponds to the second inner electrode. The first measurement pump cell 50 corresponds to the second pump cell. The third internal cavity 61 corresponds to the third chamber. The second measurement electrode 44 corresponds to the third inner electrode. The second measurement pump cell 41 corresponds to the third pump cell. The outer pump electrode 23 corresponds to the first outer electrode, the second outer electrode, and the third outer electrode. The reference electrode 42 corresponds to the reference electrode. The main pump control processing corresponds to the first pump cell control processing. The first measurement pump control processing corresponds to the second pump cell control processing. The second measurement pump control processing corresponds to the third pump cell control processing. The pump current Ip1 corresponds to the second pump current. The pump current Ip2 corresponds to the third pump current.

According to the gas sensor 100 of the present embodiment described in detail above, the sensor element 101 is configured such that the ratio Ih/Ir of the limiting current Ih to the limiting current Ir is 0.44 or less, whereby the responsiveness of water concentration measurement can be improved. Moreover, the sensor element 101 is configured such that the ratio Ic/Ir of the limiting current Ic to the limiting current Ir is 0.24 or less, whereby the responsiveness of carbon dioxide concentration measurement can be improved.

Further, the limiting current Ih is 0.3 mA or less, whereby the decrease in the accuracy of water concentration measurement can be suppressed. The limiting current Ic is 0.3 mA or less, whereby the decrease in the accuracy of carbon dioxide concentration measurement can be suppressed.

It should be noted that the present invention is not limited to the present embodiment described above in any way, and it goes without saying that the present invention can be implemented in various modes as long as they fall within the technical scope of the present invention.

For example, in the embodiment described above, Ih/Ir was 0.44 or less and Ic/Ir was 0.24 or less, but it suffices that either Ih/Ir be 0.44 or less or Ic/Ir be 0.24 or less. If Ih/Ir is 0.44 or less, the responsiveness of measuring the water concentration can be improved. If Ic/Ir is 0.24 or less, the responsiveness of measuring the carbon dioxide concentration can be improved.

In the embodiment described above, the control device 95 performed both the water concentration measurement processing and the carbon dioxide concentration measurement processing, but only one of the water concentration measurement processing and the carbon dioxide concentration measurement processing may be performed. When the control device 95 performs only the water concentration measurement processing, it suffices that Ih/Ir be 0.44 or less, and Ic/Ir need not be 0.24 or less. When the control device 95 performs only the carbon dioxide concentration measurement processing, it suffices that Ic/Ir be 0.24 or less, and Ih/Ir need not be 0.44 or less. Note that even when the control device 95 does not perform the water concentration measurement processing, it is preferable to perform the first measurement pump control processing. Likewise, even when the control device 95 does not perform the carbon dioxide concentration measurement processing, it is preferable to perform the second measurement pump control processing.

In the embodiment described above, the sensor element 101 included the porous protective layer 90, but the porous protective layer 90 need not be included. Note that, because the diffusion resistance of the porous protective layer 90 is smaller than the diffusion resistance of the measurement gas flow path (for example, the respective diffusion resistances of the first to fourth diffusion rate-limiting sections 11, 13, 30, and 60), the presence or absence of the porous protective layer 90 has little effect on the values of the limiting currents Ir, Ih, and Ic.

In the above-described embodiment, the outer pump electrode 23 plays a role as the first outer electrode to be paired with the inner pump electrode 22 of the main pump cell 21, a role as the second outer electrode to be paired with the first measurement electrode 51 of the first measurement pump cell 50, and a role as the third outer electrode to be paired with the second measurement electrode 44 of the second measurement pump cell 41. In other words, the first to third outer electrodes are configured as a common outer pump electrode 23. However, the present invention is not limited thereto. For example, two of the first to third outer electrodes may be configured as the common outer pump electrode, and the remaining one may be disposed on an outer surface of the element body 102 as an electrode independent of the outer pump electrode 23 so as to be in contact with the measurement gas. Alternatively, the first to third outer electrodes may each be disposed on an outer surface of the element body 102 as independent electrodes so as to be in contact with the measurement gas.

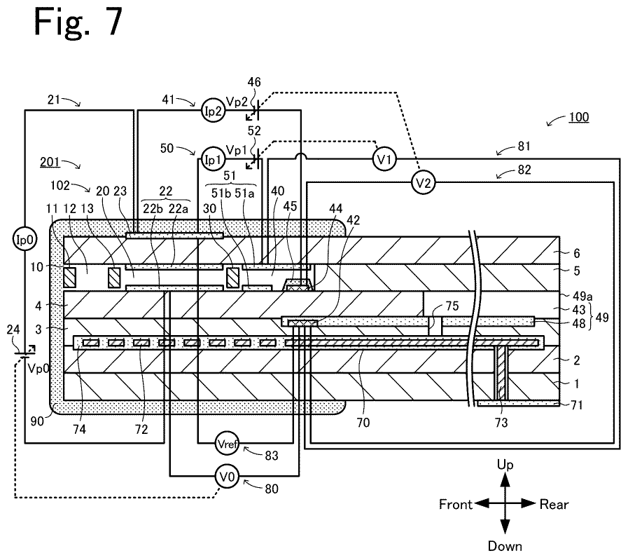

In the above-described embodiment, the sensor element 101 of the gas sensor 100 includes the first internal cavity 20, the second internal cavity 40, and the third internal cavity 61. However, the present invention is not limited thereto. For example, as shown in FIG. 7, a sensor element 201 according to a modification may not include the third internal cavity 61. In the sensor element 201 according to a modification shown in FIG. 7, between the lower surface of the second solid electrolyte layer 6 and the upper surface of the first solid electrolyte layer 4, the following components are adjacently formed and in communication with each other, in the order listed: a gas inlet 10; a first diffusion rate-limiting section 11; a buffer space 12; a second diffusion rate-limiting section 13; a first internal cavity 20; a third diffusion rate-limiting section 30; and a second internal cavity 40. In addition, the second measurement electrode 44 is disposed on the upper surface of the first solid electrolyte layer 4 within the second internal cavity 40. The second measurement electrode 44 is covered with a fourth diffusion rate-limiting section 45, which is a film made of a porous ceramic material such as alumina (Al₂O₃). The fourth diffusion rate-controlling section 45, similarly to the fourth diffusion rate-controlling section 60 of the above-described embodiment, serves to impart a predetermined diffusion resistance to the measurement gas in the second internal cavity 40 and guide the measurement gas to the second measurement electrode 44. Furthermore, the fourth diffusion rate-limiting section 45 also functions as a protective film for the second measurement electrode 44. The ceiling electrode portion 51a of the first measurement electrode 51 extends directly above the second measurement electrode 44. Even with such a configuration of the sensor element 201, the carbon dioxide concentration Ccd can be measured based on the pump current Ip2 flowing through the second measurement pump cell 41, similarly to the embodiment described above. In the sensor element 201 of FIG. 7, the region around the second measurement electrode 44 functions as the third chamber. That is, the area around the second measurement electrode 44 serves the same role as the third internal cavity 61.

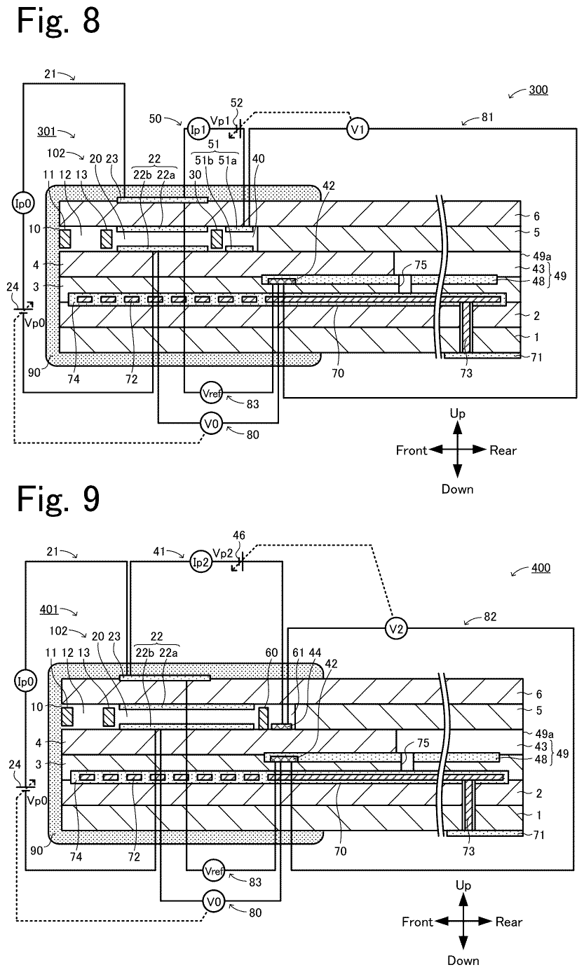

In the embodiment described above, the sensor element 101 included the first internal cavity 20, the second internal cavity 40, and the third internal cavity 61; however, such as a gas sensor 300 according to a modification shown in FIG. 8, the sensor element may be configured without the third internal cavity 61. The sensor element 301 of this gas sensor 300 does not include the fourth diffusion rate-limiting section 60, the third internal cavity 61, the second measurement electrode 44, the second measurement pump cell 41, or the second-measurement-pump-control oxygen-partial-pressure detection sensor cell 82 of the embodiment described above. Therefore, the control device for controlling this sensor element 301 is different from the control device 95 and does not include the variable power supply 46. Even in this gas sensor 300, the water concentration in the measurement gas can be measured by the control device performing the main pump control processing, the first measurement-pump control processing, and the water concentration measurement processing. If the ratio Ih/Ir of the limiting current Ir for the pump current Ip0 to the limiting current Ih for the pump current Ip1 is 0.44 or less, the responsiveness of measuring the water concentration can be improved. Note that, in this gas sensor 300, it is preferable to set the target value V0* of the main pump control processing to a value such that water in the measurement gas is reduced while the reduction of carbon dioxide is suppressed. Since carbon dioxide is harder to reduce than water, by setting the target value V0* lower than the target value V0* in the embodiment described above (i.e., to a value that sets the oxygen concentration in the first internal cavity 20 to a predetermined concentration which is higher than the oxygen concentration in the first internal cavity 20 by the main pump control processing in the above embodiment), it is possible to reduce water in the first internal cavity 20 while suppressing the reduction of carbon dioxide. Alternatively, the gas sensor 300 may be used for applications that measure the water concentration in a measurement gas that does not contain carbon dioxide.

In the embodiment described above, the sensor element 101 included the first internal cavity 20, the second internal cavity 40, and the third internal cavity 61; however, such as a gas sensor 400 according to a modification shown in FIG. 9, the sensor element may be configured without the second internal cavity 40. The sensor element 401 of this gas sensor 400 does not include the third diffusion rate-limiting section 30, the second internal cavity 40, the first measurement electrode 51, the first measurement pump cell 50, or the first-measurement-pump-control oxygen-partial-pressure detection sensor cell 81 of the embodiment described above. In the measurement-gas flow path of the sensor element 401, the first internal cavity 20, the fourth diffusion rate-limiting section 60, and the third internal cavity 61 communicate in this order. Therefore, the control device for controlling this sensor element 401 is different from the control device 95 and does not include the variable power supply 52. Even in this gas sensor 400, the carbon dioxide concentration in the measurement gas can be measured by the control device performing the main pump control processing, the second measurement pump control processing, and the carbon dioxide concentration measurement processing. If the ratio Ic/Ir of the limiting current Ir for the pump current Ip0 to the limiting current Ic for the pump current Ip2 is 0.24 or less, the responsiveness of measuring the carbon dioxide concentration can be improved. Note that it is preferable to use this gas sensor 400 for applications to measure the carbon dioxide concentration in a measurement gas that does not contain water.

In the above-described embodiment, the element body 102 of the sensor element 101 is formed as the laminated body including multiple solid electrolyte layers (layers 1 to 6). However, the present invention is not limited thereto. The element body of the sensor element 101 only needs to include at least one oxygen ion-conductive solid electrolyte layer, and have a measurement gas flow path therein. For example, in FIG. 1, layers 1 to 5, except for the second solid electrolyte layer 6, may be structural layers made of materials other than solid electrolyte (e.g., layers made of alumina). In such a case, each electrode included in the sensor element 101 may be disposed on the second solid electrolyte layer 6. For instance, the second measurement electrode 44 shown in FIG. 1 may be disposed on a lower surface of the second solid electrolyte layer 6. Furthermore, the reference gas introduction space 43, which is formed in the first solid electrolyte layer 4, may instead be formed in the spacer layer 5. Likewise, the reference gas introduction layer 48, located between the first solid electrolyte layer 4 and the third substrate layer 3, may instead be provided between the second solid electrolyte layer 6 and the spacer layer 5. In addition, the reference electrode 42 may be provided on the lower surface of the second solid electrolyte layer 6 at a position downstream of the third internal cavity 61.

What is claimed is:

1. A gas sensor comprising a sensor element and a control device, and configured to measure a water concentration and/or a carbon dioxide concentration in a measurement gas,
wherein the sensor element includes:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on an outer surface of the element body;
a second pump cell including a second inner electrode disposed in a second chamber located downstream of the first chamber of the measurement gas flow path, and a second outer electrode disposed on an outer surface of the element body; and
a third pump cell including a third inner electrode disposed in a third chamber located downstream of the second chamber of the measurement gas flow path, and a third outer electrode disposed on an outer surface of the element body;
the control device performs:
a first pump cell control processing in which oxygen is pumped out from around the first inner electrode to around the first outer electrode by controlling the first pump cell, thereby reducing water and carbon dioxide in the measurement gas in the first chamber;
a second pump cell control processing in which oxygen is pumped from around the second outer electrode to around the second inner electrode by controlling the second pump cell, thereby oxidizing hydrogen generated by the reduction of water in the first chamber in the second chamber; and
a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and,
in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], a limiting current at the time the second pump cell pumps out oxygen from around the second inner electrode to around the second outer electrode is defined as a limiting current Ih [mA], and a limiting current at the time the third pump cell pumps out oxygen from around the third inner electrode to around the third outer electrode is defined as a limiting current Ic [mA],
the control device performs a water concentration measurement processing for measuring the water concentration in the measurement gas based on a second pump current flowing through the second pump cell by the second pump cell control processing, and the sensor element satisfies that a ratio Ih/Ir of the limiting current Ih to the limiting current Ir is 0.44 or less; and/or the control device performs a carbon dioxide concentration measurement processing for measuring the carbon dioxide concentration in the measurement gas based on a third pump current flowing through the third pump cell by the third pump cell control processing, and the sensor element satisfies that a ratio Ic/Ir of the limiting current Ic to the limiting current Ir is 0.24 or less.

2. The gas sensor according to claim 1,
wherein the control device performs the water concentration measurement processing, and the limiting current Ih is 0.3 mA or less; and/or the control device performs the carbon dioxide concentration measurement processing, and the limiting current Ic is 0.3 mA or less.

3. The gas sensor according to claim 1,
wherein the control device performs the water concentration measurement processing and the carbon dioxide concentration measurement processing, and
the sensor element satisfies that the ratio Ih/Ir is 0.44 or less and the ratio Ic/Ir is 0.24 or less.

4. A gas sensor comprising a sensor element and a control device, and configured to measure a carbon dioxide concentration in a measurement gas,
wherein the sensor element includes:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on an outer surface of the element body; and
a third pump cell including a third inner electrode disposed in a third chamber located downstream of the first chamber of the measurement gas flow path, and a third outer electrode disposed on an outer surface of the element body;
the control device performs:
a first pump cell control processing in which oxygen is pumped out from around the first inner electrode to around the first outer electrode by controlling the first pump cell, thereby reducing carbon dioxide in the measurement gas in the first chamber;
a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and
a carbon dioxide concentration measurement processing for measuring the carbon dioxide concentration in the measurement gas based on a third pump current flowing through the third pump cell by the third pump cell control processing; and
in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the third pump cell pumps out oxygen from around the third inner electrode to around the third outer electrode is defined as a limiting current Ic [mA], a ratio Ic/Ir of the limiting current Ic to the limiting current Ir is 0.24 or less.

5. A sensor element for measuring a water concentration and/or a carbon dioxide concentration in a measurement gas, the sensor element comprising:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on an outer surface of the element body;
a second pump cell including a second inner electrode disposed in a second chamber located downstream of the first chamber of the measurement gas flow path, and a second outer electrode disposed on an outer surface of the element body; and
a third pump cell including a third inner electrode disposed in a third chamber located downstream of the second chamber of the measurement gas flow path, and a third outer electrode disposed on an outer surface of the element body;
wherein, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], a limiting current at the time the second pump cell pumps out oxygen from around the second inner electrode to around the second outer electrode is defined as a limiting current Ih [mA], and a limiting current at the time the third pump cell pumps out oxygen from around the third inner electrode to around the third outer electrode is defined as a limiting current Ic [mA], a ratio Ih/Ir of the limiting current Ih to the limiting current Ir is 0.44 or less and/or a ratio Ic/Ir of the limiting current Ic to the limiting current Ir is 0.24 or less.

6. A sensor element for measuring a carbon dioxide concentration in a measurement gas, the sensor element comprising:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on an outer surface of the element body; and
a third pump cell including a third inner electrode disposed in a third chamber located downstream of the first chamber of the measurement gas flow path, and a third outer electrode disposed on an outer surface of the element body;
wherein, in the state of an inlet of the measurement gas flow path exposed to an atmosphere of a model gas in which nitrogen is a base gas and an oxygen concentration is 20%, when a limiting current at the time the first pump cell pumps out oxygen from around the first inner electrode to around the first outer electrode is defined as a limiting current Ir [mA], and a limiting current at the time the third pump cell pumps out oxygen from around the third inner electrode to around the third outer electrode is defined as a limiting current Ic [mA], a ratio Ic/Ir of the limiting current Ic to the limiting current Ir is 0.24 or less.
